(19) United States
(12) Patent Application Publication
(10) Pub. No.: US 2012/0213666 A1
(43) Pub. Date: Aug. 23, 2012

(54) APPARATUS AND METHOD FOR DECONTAMINATION BY MEANS OF SEQUENTIAL HYDROGEN PEROXIDE FOGGING WITH A VEIW TO CREATING DRY MIST

(76) Inventor: Frederic Goessens, Vieillevigne (FR)

(21) Appl. No.: 13/502,305

(22) PCT Filed: Oct. 15, 2010
(86) PCT No.: PCT/FR2010/000686

§ 371 Date: Apr. 16, 2012

(30) Foreign Application Priority Data
Oct. 16, 2009 (FR) 0904972

(51) Int. Cl. A61L 2/18

(52) U.S. Cl. 422/28

(57) ABSTRACT

The invention mainly relates to an apparatus for decontaminating an indoor space (1), said apparatus comprising a means for spreading a liquid treatment material starting with fogging of a predetermined amount (Q) of said treatment liquid in the space (V) of the latter. The decontamination apparatus of the invention is characterized in that it comprises a means for controlling the spreading means, said controlling means being capable of stopping and resuming the fogging of said liquid material a plurality of times during a single treatment cycle. The present invention also relates to a decontamination method implementing said apparatus.

The present invention relates to a treatment method for providing a decontamination by fogging a liquid disinfecting material onto surfaces to be treated, and in particular onto the walls of an inside space and the different apparatuses and instruments contained therein.

By fogging, it will be meant an emission of a liquid material the spreading of which will be homogeneous in a given volume as a “dry mist”, that is not forming apparent condensation onto the surfaces.

It is known that bacterial agents which cause the contamination and are air-borne in an indoor space tend to settle down on different surfaces and objects contained therein. It is known besides, conversely, that the bacterial agents which grow on objects and on the walls of an inside space (for example operating rooms, clean rooms or various treatment rooms, etc.) tend to be borne in the atmosphere. These indoor spaces are thus in a permanent exchange situation between the walls and objects on the one hand and the atmosphere on the other hand.

It is understood under these conditions that it is essential that global decontamination should be applied to the atmosphere on the one hand and to various surfaces of the inside room on the other hand.

It has been observed that this kind of treatment has several drawbacks. First, the size of the drops formed is relatively high (in the order of 80 to 200 μm for an air flow rate of 3-5 mL air per minute), so that these drops become deposited by a mere gravity effect onto the surfaces close to their fogging place which, of course, is unsatisfactory given that the surfaces distant from the fogging injector are free from treatment.

Secondly, due to the size of the drops, they tend to be pooled and form at the surface of the walls and objects of the inside room a wet film, or even liquid pools. This condensation results in generating a chemical attack of these contact surfaces, so that it is necessary to periodically rework the surface of the walls of the inside room and to replace the objects and apparatuses which, due to the chemical attack they underwent, turn out to be unsuitable for the use intended therefore.

Such a phenomenon has been dramatically minimized by a kind of fogging provided in FR patent 2 859 650 on behalf the applicant, which enables the fractionation of sprayed drops to be improved by resorting to an injection device enabling fine droplets to be obtained the dimensions of which are in the order of 2 μm-20 μm and which have the property to be borne in the entire volume of the indoor space and to be deposited onto the walls and objects contained therein without aggregating together so that they form a continuous film; thus generated mist being called “dry mist”.

However, one of the difficulties inherent to any kind of fogging treatment is that it requires, apparently contradictorily, to be both sufficiently active to be able to provide its decontamination action for a relatively short time and, in the same time, not to cause a chemical attack of walls and objects contained in the indoor space subjected to the treatment.

The object of the present invention is to provide a treatment method and a treatment apparatus enabling the integrity of surfaces with which the treatment liquid is caused to contact to be respected, as well as to improve the efficiency of the treatment performed.

Thus, one object of the present invention is an apparatus for decontaminating an indoor space, comprising a means for spreading a liquid treatment material starting with fogging of a predetermined amount of said treatment liquid in the space of the latter, characterised in that it comprises a means for controlling the spreading means, said controlling means being capable of stopping and resuming the fogging of said liquid material a plurality of times during a single treatment cycle.

Preferentially, the decontamination apparatus according to the invention:
will comprise means for measuring the indoor space atmosphere concentration of a material, called controlled material, which is at least correlated with the treatment liquid,
it will comprise means for slaving the spreading means to the measurement means,
the means for controlling the spreading means are capable of: a) providing the fogging until a stage forming a maximum concentration of said controlled material is reached in said indoor space, b) adjusting the fogging to maintain this concentration level of said controlled material in the indoor space for a given duration between zero and five minutes and preferentially close to 1 minute, c) stopping the fogging until a stage forming a minimum concentration of said controlled material is reached, d) repeating this cycle until the determined amount of treatment liquid has been distributed.

In one embodiment of the invention, the means for controlling the spreading means will enable the stage forming the maximum concentration of said controlled material to be reached from concentration values lying under said maximum.

In another embodiment of the invention, the means for controlling the spreading means will enable the stage forming the maximum concentration of said controlled material to be reached from concentration values lying above said maximum.

According to the invention, the controlled material will be of the same nature as the treatment liquid; the latter can in particular consist of an aqueous hydrogen peroxide solution. The weight concentration of the latter can in particular be between 3% and 9% and preferentially in the order of 6%.

Besides, the measurement means can comprise at least one sensor for measuring the indoor space atmosphere concentration of said controlled material. They can also comprise a sensor for detecting an insufficient amount of treatment liquid, such sensor being provided in the indoor space, in particular at a critical area thereof.

The apparatus will advantageously comprise means capable of communicating with said sensor by a remote connection, in particular of the “Bluetooth” type.

The indoor space atmosphere maximum concentration of said controlled material will be between 90% and 97% and preferentially in the order of 95%. Besides, the indoor space atmosphere minimum concentration of said controlled material will be between 75% and 95% and preferentially in the order of 90%.

The measurement means could be provided on the apparatus, preferentially on a part opposite to the direction of ejection of the treatment liquid.

One object of the present invention is also to provide an assembly for decontaminating an indoor space by fogging in the volume thereof a predetermined amount of a liquid treatment material, characterised in that the amount of treatment liquid is provided by an assembly of several apparatuses of the above-mentioned type.

One object of the present invention is also to provide a method for decontaminating an indoor space by fogging in the volume thereof a predetermined amount of a liquid treatment material, characterised in that the fogging of said liquid material is stopped and resumed a plurality of times during a single treatment cycle.

Preferentially, this decontamination method will comprise the steps of:
providing the fogging until a maximum concentration of a controlled material which is at least correlated with the treatment liquid is reached in said indoor space,
setting the fogging to maintain this concentration level of said controlled material in the indoor space during a maintenance duration between zero and five minutes and preferentially close to one minute,
stopping the fogging until a minimum concentration of said controlled material is reached,
repeating this cycle until the predetermined amount of treatment liquid has been distributed.

In a particularly interesting embodiment of the invention, the maintenance duration will be equal to zero.

In one embodiment of the invention, the controlled material and the treatment liquid will be of the same nature.

In one alternative of the invention, the fogging step during which the indoor space concentration of said treatment material is brought to the maximum concentration is provided by making at least one concentration peak; several of them could form an intermediate stage.

It will be described hereinafter, by way of non-limiting example, an embodiment of the present invention in reference to the appended drawing wherein:

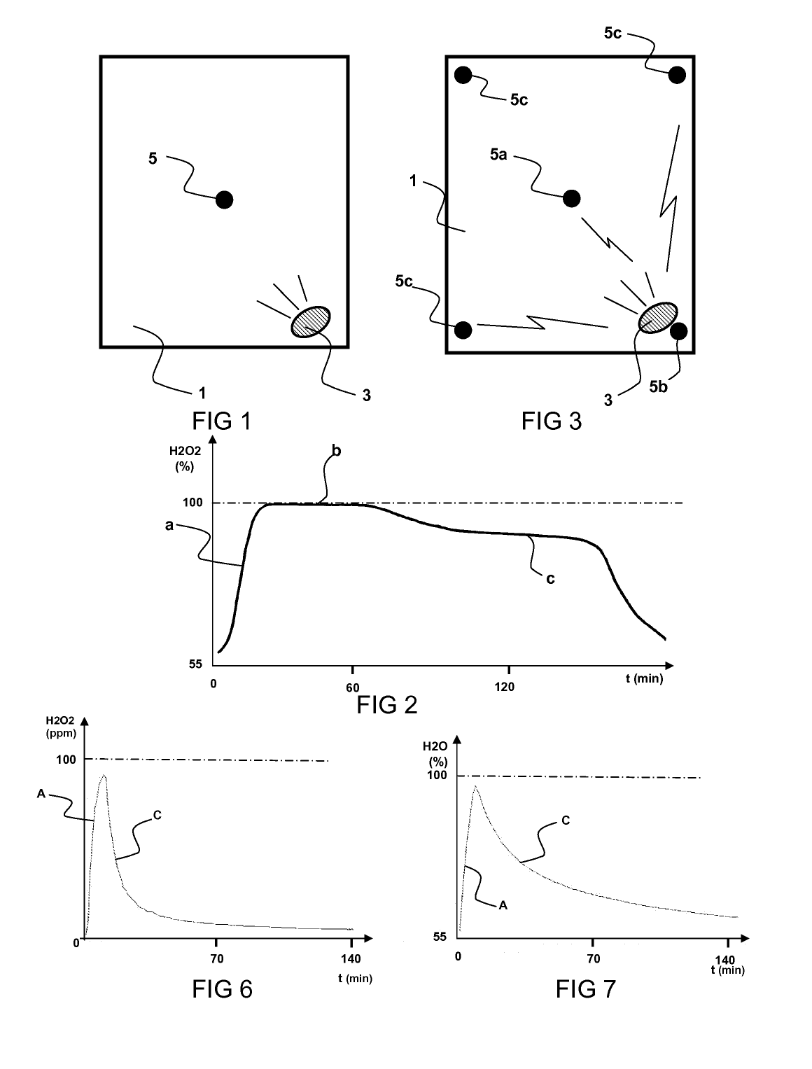

FIG. 1 is a schematic view of an indoor space to be decontaminated by means of an apparatus according to the state of prior art,

FIG. 2 is a schematic graph represented the concentration of treatment material which exists in the indoor space to be decontaminated during a treatment according to the state of prior art,

FIG. 3 is a schematic view of an indoor space to be decontaminated by means of an apparatus and the method according to the invention,

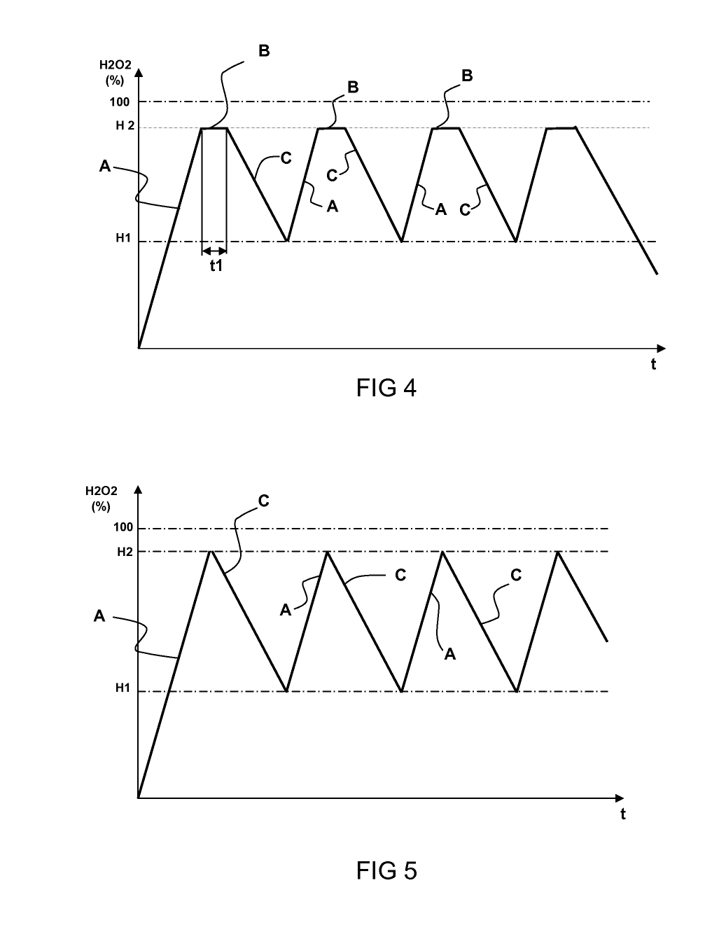

FIG. 4 is a schematic and theoretical graph representing the concentration of treatment material in the indoor space to be decontaminated throughout an operation according to the invention,

FIG. 5 is a schematic and theoretical graph of an alternative embodiment of the invention represented in FIG. 4,

FIGS. 6 and 7 represent the respective concentrations of hydrogen peroxide and water steam in an indoor space wherein a hydrogen peroxide solution has been spread,

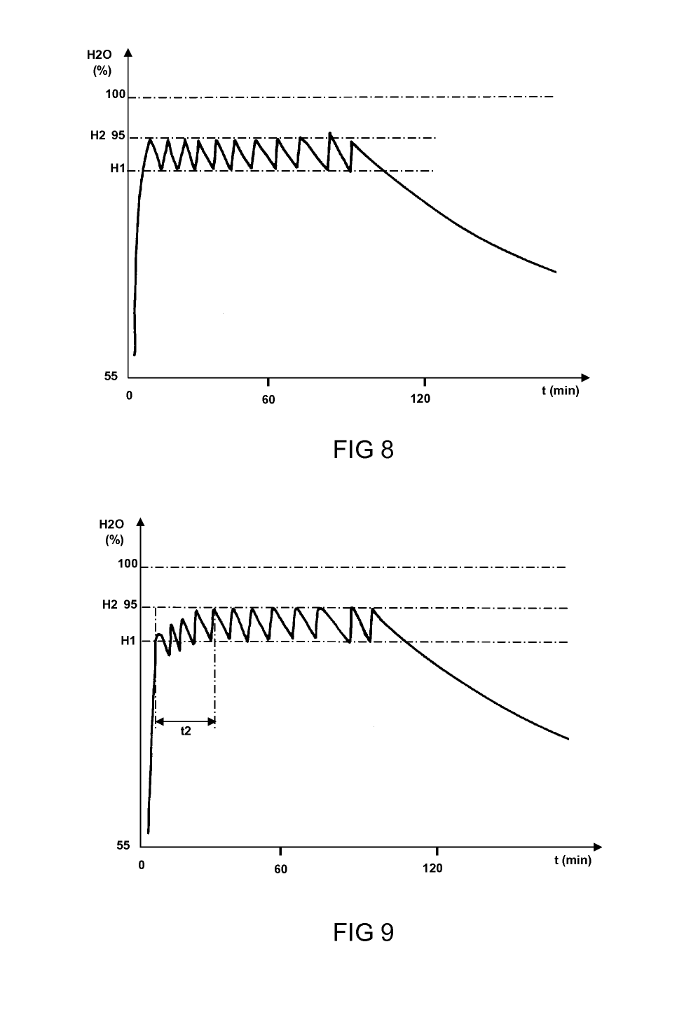

FIGS. 8 and 9 are graphs representing throughout a decontamination operation according to the invention the concentration of water steam in the indoor space to be treated, which is measured with two sensors provided in the centre of the indoor space and behind the treatment apparatus respectively,

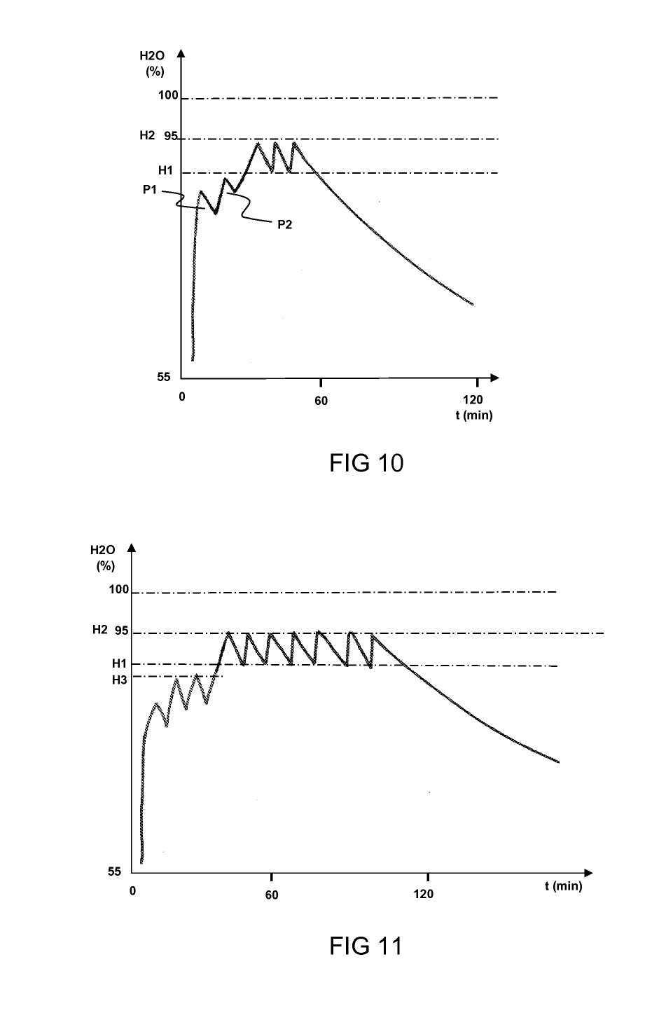

FIGS. 10 to 13 are graphs illustrating alternative embodiments of the present invention.

FIG. 1 show an indoor space 1 the decontamination of which is desired to be provided by means of an apparatus 3 in accordance with the state of prior art, and which is capable of spreading into this indoor space, a liquid treatment material, consisting in particular of an aqueous hydrogen peroxide solution having a concentration in the order of 6% by weight and with a flow rate in the order of 30 ml/min. In a known manner, the treatment material flow rate is adjusted as a function of the volume V of the indoor space 1. Therefore, it can be necessary, in the case of high volume indoor spaces, to provide several treatment apparatuses therein.

In the centre of the indoor space 1, is provided a sensor 5 able to measure the hydrogen peroxide content thereof and a treatment operation of the indoor space 1 has been performed. FIG. 2 represents the curve showing the variation in the hydrogen peroxide content in the indoor space 1 during this treatment.

It can be seen therefore that a conventional treatment cycle is divided into several phases, that is:
a first phase a during which the hydrogen peroxide concentration quasi linearly increases until it reaches a stage b representing the treatment material saturation,
a second phase forming the stage b during which the treatment material is still spread into the indoor space 1 and during which it condensed onto the contact surfaces such as the walls and various apparatuses contained therein,
a third phase c during which any spreading is stopped, so that the hydrogen peroxide concentration gradually decreases.

It is known that the condensation which occurs on the contact surfaces of the objects during the treatment has a destructive action on the latter which forces their restoration or periodical replacement.

It has been set by the applicant that spreading by alternately fogging the treatment liquid, any noticeable condensation and, consequently, any chemical attack of the contact surfaces as well could be avoided.

By alternated spreading, it will be meant a spreading which is stopped and resumed a plurality of times during a single treatment cycle so as to form concentration peaks, regardless of whether these peaks form stages as represented in FIG. 4, whether these peaks are regular and periodical as represented in FIG. 5, whether they are fully contained between stages forming maximum H2 and/or minimum H1 values as represented in FIG. 8, or whether they are only partially contained between these values as represented in FIGS. 10-13.

Preferentially, the maximum hydrogen peroxide content will be restricted inside the indoor space to a maximum value H2 slightly lower than saturation, that is between 90% and 97% and preferentially in the order of 95%, and this content will be periodically allowed to decrease to a minimum value H1 in particular between 75% and 95% and preferentially in the order of 90%.

To do this, according to the invention and as represented in FIG. 3, the fogging apparatus 3a comprises means for coupling and slaving with at least one detector or sensor 5a capable of measuring the hydrogen peroxide content in the indoor space 1 during the treatment. These coupling means can be made by a simple wiring or, more practically, by remote transmission means, in particular of the so-called “Bluetooth” type. The values measured by the sensor 5a are sent back to the apparatus 3a where they are processed by a logical computing unit.

As represented in FIG. 4, an alternate spreading by fogging is provided according to the invention. The fogging (part A of the curve) is started with a flow rate in the order of that used according to the state of prior art, that is 30 ml/min, until the hydrogen peroxide content in the indoor space reaches a maximum value H₂, and then the control logic decreases the fogging flow rate so as to maintain constant said content to this maximum value for a time t1 (part B of the curve). The control logic then stops fogging, such that the hydrogen peroxide content in the indoor space 1 decreases (part C of the curve) until it reaches the minimum value H₁. The control logic then provides the resuming of fogging under the same conditions as the initial fogging (part A of the curve).

An alternate fogging thus proceeds until the desired amount of treatment material Q to be distributed during the operation has been spread in the indoor space.

It has been observed that, due to its alternated character, the spreading mode by fogging according to the invention avoids any apparent condensation of the treatment liquid on the contact surfaces. The optimum value of the maintenance time t1 for given fogging conditions could be empirically determined by decreasing the same when a condensation comes to be detected on a contact surface. Tests carried out by the applicant have shown that the time is generally between 0 and 5 minutes.

Advantageously, in particular when the contact surfaces belong to fragile objects, the maintenance time t1 of the indoor space content of treatment material at the maximum value H₂ could be reduced to zero. Under these conditions, as soon as the treatment material content reaches the maximum value H2, the control logic will stop the fogging until the same reaches the minimum value H1, to resume it thereafter, as represented in the curve of FIG. 5 so as to provide an alternate fogging.

Preferentially, the measurement of the indoor space concentration of treatment material will be carried out by means of several measurement probes or sensors 5a, 5b, 5c which will be distributed in the indoor space 1 to be treated, at positions depending on the specific geometry thereof, as represented in FIG. 3.

The control carried out by means of the measurement probes during the treatment operation could of course directly relate to the indoor space atmosphere content of the treatment material itself, but it could also relate to another parameter, with the proviso that the same could be correlated with the former. Thus, according to the invention, in the case where the treatment material consists of hydrogen peroxide, the measurement could be carried out on a hydrogen peroxide substitute, that is water, this material being called “controlled material” herein below.

Indeed, if the variation in the hydrogen peroxide content (FIG. 6) and the variation in hygrometry (FIG. 7) in an indoor space wherein a hydrogen peroxide solution is spread by fogging are measured and represented, it will be observed that, both during the first fogging phase (phase A) and the phase following the stoppage of fogging (phase C), that the aspects of both curves are similar.

Such an arrangement is particularly interesting in that it enables hydrogen peroxide sensitive sensors which have a high cost and demand a constant maintenance to be replaced by humidity sensors which in turn have a much lower cost and only require little maintenance.

As represented in FIG. 3, several measurement probes can be provided in the indoor space at places where an average statistical result is obtained, in particular in the middle of the indoor space (measurement probe 5a) or in the contrary in places where it is believed that the treatment material might spread with some difficulty (measurement probe 5c).

Besides, it is interesting to provide a treatment apparatus which is self-contained, that is which has its own measurement probe and does not require resorting to satellite probes such as the probes 5a and 5c represented in FIG. 3, at least when the indoor space 1 to be treated has a simple geometrical shape.

It has been set according to the invention that a probe provided at the rear part of the treatment apparatus (measurement probe 5b), in particular at the opposite of the direction of ejection of the treatment liquid, is able to provide the humidity rate measurement which will be perfectly representative of the average global rate in the indoor space.

Therefore, various treatment operations have been carried out by resorting to two measurement probes, that is a probe 5a provided in the centre of the indoor space and which is coupled through a remote connection, for example a so-called “Bluetooth” connection, with the apparatus 3a and a measurement probe 5b provided behind it and which is not coupled with the apparatus.

If the representative curves during the treatment of the humidity rate of the indoor space in the centre thereof on the one hand (FIG. 8), and behind the apparatus 3a on the other hand (FIG. 9) are compared, it can be seen that apart from a slight offset during a time t₂, the curves are perfectly similar, so that the treatment apparatus can be driven by a measurement probe coupled with the same and which is provided on its rear part, that is on a part thereof opposite to the direction of ejection.

Exemplary Embodiment

In an indoor space 1 having a volume V=40 m³, an amount Q=720 ml of a 6% by weight aqueous hydrogen peroxide solution has been alternately spread by fogging according to the invention, following the fogging profile represented in FIG. 8. The apparatus was provided at an angle of the indoor space 1 and the single humidity sensitive measurement probe 5a was provided in the centre of the indoor space.

In this example, the maximum H2 has been set to a 95% humidity rate and the minimum H1 has been set in turn to a 90% humidity rate. Besides, the maintenance duration t1 was equal to 0, that is as soon as the maximum concentration H2 was reached, the control logic of the apparatus stopped the fogging to resume it once the minimum concentration H1 was reached.

Once the operation was carried out, the presence of no condensation pool was observed both on the walls of the indoor space and on the objects and apparatuses contained therein.

If the results obtained in the method according to the invention are compared with those obtained in the conventional method according to the state of prior art, it can be seen that an amount Q of treatment liquid which is in the order of three times that usually used (240 ml for an indoor space of 40 m³) in a same volume V of the indoor space has been distributed, without causing any apparent pool and without causing neither oxidation nor deterioration in present materials or equipment.

Besides, microbial analyses have shown that the initial strain of a 3.10⁸ cfu/ml (colony forming units/ml) staphylococcus aureus stock solution, the reduction in the bacterial load is log 10 thus showing a very efficient decontamination which turns out to be higher than that obtained in conventional methods according to the state of prior art.

The apparatus and method according to the invention can be substantially used in two ways, namely on the one hand in a preventive manner and on the other hand in a curative manner which respectively corresponds to a low recontamination risk and a sharp recontamination risk, in this case by varying the amount Q of treatment liquid emitted and spread by the apparatus. By way of example in a preventive manner, an amount Q of 8 ml/m³ could be chosen whereas in a curative manner, an amount Q of 18 ml/m³ will be chosen. It is understood that under these conditions, for an apparatus having a given flow rate, the preventive treatment will be quicker, thereby making up a so-called “short cycle” treatment cycle.

The present invention thus enables, without creating an apparent condensation on exposed contact surfaces, and consequently without causing corrosion, a volume Q of treatment liquid much higher than that according to the state of prior art to be spread, which also enables the efficiency of the treatment to be increased.

In an alternative embodiment of the present invention, the first step of the fogging method during which the room concentration of said controlled material is brought to the maximum concentration H2 could be provided not continuously as previously described, but by making one or more concentration peaks P1, P2, as represented in FIG. 10.

In an embodiment of this alternative, represented in FIG. 11, several of these peaks could form one or more intermediate stages H3.

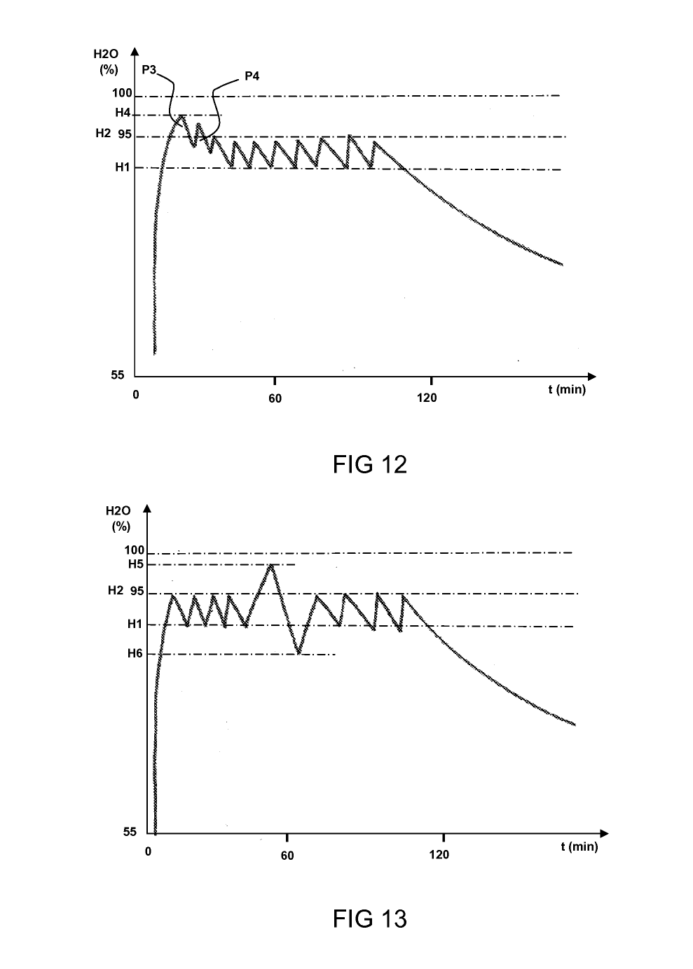

In another embodiment of the present invention, represented in FIG. 12, the first step of the method could bring the room concentration of said controlled material to a value higher that the stage consisting of the maximum H2, that is a value H4 in the figure. It could then lower back by an alternate fogging to the maximum value H2, either directly, or by forming peaks P3 and P4. The following of the fogging could then continue to alternately proceed between stages forming the maximum H2 and minimum H1 values until the end of the treatment cycle.

Finally, during this cycle, as represented in FIG. 13, there could be a raise to a concentration value (H5 in the figure) higher than the stage of the maximum value H2 or a drop to a concentration value (H6 in the figure) lower than the stage of the minimum value H1.

It can be seen according to the invention that, given that the fogging spreading of the treatment liquid is alternately carried out during a treatment cycle, the formation of liquid pools thereof which are the main cause for degradation phenomena of walls of the volumes treated as well as objects contained in the same can avoided, while improving the decontamination quality at the same time.

1-15. (canceled)

16. A method for decontaminating an indoor space (1) by fogging in the volume (V) thereof a predetermined amount (Q) of a liquid treatment material, characterised in that the fogging of said liquid material is stopped and resumed a plurality of times during a single treatment cycle.

17. The method for decontaminating an indoor space (1) according to claim 16, characterised in that it comprises the following steps of:
providing the fogging until a maximum concentration (H2) of a controlled material which is at least correlated with the treatment liquid is reached in said indoor space (1),
setting the fogging to maintain this concentration level of said controlled material in the indoor space (1) during a maintenance duration (t1) between zero and five minutes and preferentially close to one minute,
stopping the fogging until a minimum concentration (H1) of said controlled material is reached,
repeating this cycle until the predetermined amount (Q) of treatment liquid has been distributed.

18. The method for decontaminating an indoor space (1) according to claim 17, characterised in that the maintenance duration (t1) is equal to zero.

19. The method for decontaminating an indoor space (1) according to claim 17, characterised in that the controlled material and the treatment liquid are of the same nature.

20. The method for decontaminating an indoor space (1) according to claim 17, characterised in that the fogging step during which the indoor space (1) concentration of said treatment material is brought to the maximum concentration (H2) is provided by making at least one concentration peak (P1, P2).

21. The method for decontaminating an indoor space (1) according to claim 20, characterised in that at least two concentration peaks (P1, P2) forming an intermediate stage (H3) are formed.
